(19) United States
(12) Patent Application Publication
(10) Pub. No.: US 2025/0058322 A1
(43) Pub. Date: Feb. 20, 2025

(54) DEVICE OF COLLECTION OF BIOLOGICAL SAMPLES, CONTAINER FOR THIS DEVICE AND METHOD OF COLLECTION OF BIOLOGICAL SAMPLES

(71) Applicant: COPAN ITALIA S.p.A., Brescia (IT)

(72) Inventors: Giorgio Martello, Brescia (IT); Lorenzo Fumagalli, Brescia (IT); Cristiano Sabelli, Brescia (IT)

(21) Appl. No.: 18/721,617

(22) PCT Filed: Dec. 19, 2022
(86) PCT No.: PCT/IB2022/062460

§ 371 Date: Jun. 18, 2024

(30) Foreign Application Priority Data
Dec. 20, 2021 (IT) 102021000031799

(51) Int. Cl. B01L 3/00; A61B 10/00

(52) U.S. Cl. CPC B01L 3/508; A61B 10/0045; B01L 2200/0689; B01L 2300/02

(57) ABSTRACT

A container for a device for collecting and transferring samples of biological material, comprising a main body destined to contain and/or support at least temporarily at least a device for collecting and transferring samples of biological material; and a graduated scale, comprising a plurality of measurement markings configured to, and destined to, supply an at least visual and/or tactile indication of a measurement of a distance; said container being configured to allow a measurement of a distance between a first portion and a second portion of the body of a subject, preferably a distance between an ear, in particular the tragus or lobe or opening of the ear canal, and an opening or end of a naris of a subject and/or between an ear, in particular the tragus or lobe or opening of the ear canal, and the philtrum of the subject by said graduated scale.

### TECHNICAL FIELD

The present disclosure refers to the field of collection of biological samples, in particular to the collection of biological samples through the introduction of suitable devices of collection within cavities of the human or animal body. The disclosure refers to a device for collecting biological samples, a container for this device and a method of collection of biological samples.

### BACKGROUND ART

Devices of collection of biological samples are traditionally used for collecting biological samples from cavities of human or animal body. In particular, among the devices for the collection of known type there are the so-called nasopharyngeal swabs, which are devices equipped with a substantially elongated body, typically having a substantially axial extension, which ends with a collection element of biological samples destined to retain a biological sample substantially fluid. Other types of devices for the collection of biological samples are destined to be introduced in cavities different with respect to the nose, for example in the mouth, in the vagina or anus, in order to collect the biological samples here present, such as for example epithelial cells, secretions, etc. . . .

The nasopharyngeal swabs are typically introduced in the nasal cavity through the opening of a naris, and are pushed in the nasal cavity until typically reaching a mucosa present in the nasopharynx, which is substantially placed in correspondence of the rear portion of the nasal cavity, for collecting biological samples, such as epithelial cells, secretions, etc. . . . , of the upper respiratory tracts.

After the collection, the nasopharyngeal swab is extracted from the nose and the collected sample can subsequently be placed for example in a reagent, to determine the presence or absence of determined pathogens, or subjected to other types of analysis.

Since the first months of 2020, the rapid spread of the SARS-COV2 virus responsible for COVID-19 disease has led to a great diffusion of the use of nasopharyngeal swabs for carrying out screening and/or testing for coronavirus infection.

In the case of screening and/or testing for COVID-19, the WTO recommends carrying out nucleic acid amplification tests (NAATs) using a reverse polymerase chain reaction process, on biological samples collected generally via nasopharyngeal swabs.

It has been observed that the correct use of nasopharyngeal swabs increases the reliability of the screening and/or test. In particular, it has been observed that a proper mode of collection of the biological sample, represented by epithelial cells, secretions, or other analytes collected from the upper respiratory tracts, allows to improve the reliability of the screening and/or test, in particular by mitigating the number of false negatives. False negatives are those tests carried out through nasopharyngeal swabs that are negative (i.e. report an apparent absence of traces of SARS-CoV2) although the subject is actually infected. A presence of a large quantity of false negatives determines—at least indirectly—an increase in the spread of the contagion, since a subject who should be placed in isolation is instead free to move around. False negatives are particularly insidious in the case of asymptomatic infection.

It is observed that screening and/or testing for COVID-19 is carried out on people of all ages, both children and adults.

Prior to the spread of SARS-CoV2 virus, nasopharyngeal swabs were used almost exclusively by highly specialized personnel. Nowadays, screening and/or testing with nasopharyngeal swabs are often no longer carried out solely by specialized medical personnel, but mostly by paramedics or pharmacy staff, whose training in this type of collection is generally sub-optimal and lower than that of a doctor. It has been observed in particular that in many cases the training of this personnel is limited to viewing informative videos.

As already noted, the reliability of the screening and/or test depends to a large extent on the correct reaching of the rear mucosa of the nasopharynx, and thus on the correct actuation of the procedure of insertion and collection of the device.

On one hand, therefore, an insufficient introduction of a nasopharyngeal swab can then determine an inadequate collection of the desired sample.

On the other hand, it has been observed that an excessive introduction of a nasopharyngeal swab can cause pain and/or trauma to the patient.

Although reference has been made to the nasopharyngeal swab, and thus to the nasal cavity, the drawbacks of an excessive or reduced introduction of a swab into another body cavity are the same.

Guidelines have therefore been proposed in the state of the art to allow an adequate collection of the sample, but these guidelines are often inhomogeneous with each other and often leave an excessive margin of interpretation and uncertainty in the operators.

It is known that the distance between the opening of the naris and the rear mucosa of the nasopharynx, which determines the optimal depth of introduction of the device for the collection, is firstly related to the age of the subject upon whom the screening and/or test by the nasopharyngeal swab is carried out, as this distance increases with increasing age of the subject and at a determined age, substantially stabilizes.

It is also known that this distance also varies depending on further factors related to the specific anatomical conformation of the different subjects and therefore results to be not predictable in a sufficiently precise way.

The purpose of the present disclosure is to provide a device for the collection, a container for devices for the collection and/or a collection method which are able to overcome the limitations of the known art and in particular to improve the reliability of the screening and/or test following the introduction of the device into a body cavity.

### SUMMARY

The Applicant has observed that the correct depth of insertion of the nasopharyngeal swab for the carrying out of screening and/or diagnostic tests to assess infections with viruses that may be found in the upper airways, be they SARS-Cov2 type viruses or others, can be estimated in a sufficiently precise way depending on the size of other parts of the patient's head to be sampled.

In particular, this correct depth can be estimated in a sufficiently precise way as a function of the distance between the tip or end of the naris and a determined part of the ear.

The object of the present disclosure is now described in some of its salient aspects, which can be combined with each other or with portions of the detailed description and/or claims.

According to the present disclosure it is first of all described a device (1) for collecting and transferring samples of biological material, comprising:
an elongated body (2), at least partially extending along a main substantially axial development direction (X) and comprising at least a gripping portion (2i) destined to be grasped by a user and at least an end portion (2e);
a collection element (3) connected to the elongated body (2) in substantial correspondence of said end portion (2e) and configured to collect and/or absorb a quantity of a sample of biological material present in a cavity of the body of a subject (20), at least the collection element (3) being configured to be introduced inside a cavity of the body of the subject (20), for example to be introduced inside the nasal cavity and/or the nasopharynx of the subject (20), or inside the mouth, vaginal or anal cavities of the subject,
   wherein the elongated body (2) comprises a graduated scale (4) provided with a plurality of measurement markings (4i) configured to, and destined to, provide an at least visual and/or tactile indication of a measurement of a distance with respect to said end portion (2e) of the elongated body and/or to a predefined portion of said collection element (3), preferably at the end of said collection element (3) opposed to the gripping portion.

According to another non-limiting aspect, the graduated scale (4) is configured and destined to provide an at least visual and/or tactile indication of a preferred and predetermined introduction depth (100) at least of the collection element (3) inside a cavity of the body of the subject (20), optionally inside the nasal cavity and/or the nasopharynx, preferably in relation to a distance (D) between a first portion and a second portion of the body of a subject (20), in particular in relation to a distance (D) between an ear (10), in particular the tragus or lobe or opening of the ear canal (10t), and an opening of the naris (11) of a subject (20) and/or between an ear (10), in particular the tragus or lobe or opening of the ear canal (10t), and the philtrum (12) of the subject (20).

According to another non-limiting aspect, the plurality of measurement markings (4i) are expressed according to a predetermined distance measurement unit.

According to another non-limiting aspect, the elongated body (2) is fixed with respect to the collection element (3).

According to another non-limiting aspect, the elongated body (2) does not slide with respect to this latter.

According to another non-limiting aspect, the plurality of measurement markings (4i) comprises at least a first and a second measurement marking (4i) reciprocally spaced for a predefined distance along said main development direction (X).

According to another non-limiting aspect, the plurality of measurement markings (4i) extends on at least part of a transversal development of the elongated body (2) and/or extends on at least part of a perimeter of the elongated body (2); optionally the plurality of measurement markings (4i) extending along the whole transversal development of the elongated body (2) and/or extending along the whole perimeter of the elongated body (2).

According to another non-limiting aspect, said at least a first and a second measurement marking (4i) extend at least partially, preferably mainly, along a substantially oblique direction, optionally along a substantially orthogonal direction, with respect to the main development direction (X).

According to another non-limiting aspect, at least a first, a second and a third measurement marking (4i) of said plurality of measurement markings (4i) are equidistant two by two.

According to another non-limiting aspect, said graduated scale (4) allows the quantitative identification of said distance between at least part of the plurality of measurement markings (4i) and said at least one between said end portion (2e) and said collection element (3).

According to another non-limiting aspect, the graduated scale (4) is at least partially etched, and/or realized through recesses or concavities, on said elongated body (2) and/or is at least partially in relief on said elongated body (2) and/or is fixed, impressed, molded, co-molded or painted on said elongated body (2).

According to another non-limiting aspect, said graduated scale (4) is realized in a material at least partially different with respect to the material with which is realized said elongated body (2), in particular wherein the elongated body (2) presents at least a first color and wherein at least part of said graduated scale (4) presents at least a second color different with respect to said first color.

According to another non-limiting aspect, the elongated body (2) comprises a central portion (2c) positioned between said gripping portion (2i) and said end portion (2e).

According to another non-limiting aspect, the graduated scale (4) extends at least partially in said central portion (2c) and/or extends at least partially in said gripping portion (2i).

According to another non-limiting aspect, the graduated scale (4) is configured and specifically destined to address, in use, the user to grasp the elongated body (2) in correspondence of at least a measurement marking (4i) indicative of said preferred and predetermined introduction depth (100).

According to another non-limiting aspect, said graduated scale (4) is provided with a plurality of numerical indications realized according to a metric scale, for example according to the decimal metric system or to the United States customary system, associated respectively in correspondence of said plurality of measurement markings (4i),

and/or the graduated scale (4) comprises at least a measurement marking (4i) positioned substantially in proximity of the collection element (3) and in particular in substantial contact with the collection element (3).

According to another non-limiting aspect, the graduated scale (4) is realized on an auxiliary element different from the main body of the container, the auxiliary element being configured to be housable inside the main body and selectively extractable to allow the measurement of the distance (D).

According to another non-limiting aspect, the device (1) has a length at least equal to 12 cm, and preferably at least equal to 13 cm or more preferably to 14 cm.

According to another non-limiting aspect, the elongated body (2) presents a substantially constant transversal section along its extension along said main development direction (X) or presents a variable transversal section along its extension along said main development direction (X); optionally the elongated body (2) presenting a transversal section substantially circular or inscribable in a circumference.

According to another non-limiting aspect, said transversal section substantially circular or said circumference has a diameter comprised between 1.5 mm and 3 mm, more preferably comprised between 2 mm and 2.5 mm.

According to another non-limiting aspect, the elongated body (2) is substantially flexible, and/or is realized at least partially in a natural or plastic material, and/or is realized through a molding process, in particular through injection molding or through co-molding, and/or the 3D printing.

According to another non-limiting aspect, the elongated body (2) is realized as a single piece.

According to another non-limiting aspect, the graduated scale (4) is integral with said elongated body (2).

According to another non-limiting aspect, said graduated scale (4) is realized integrally with said elongated body (2), in particular through said molding process, in particular injection molding or co-molding, and/or the 3D printing.

According to the present disclosure is also described a container (9) for a device (1) for collecting and transferring samples of biological material, preferably according to one or more of the aspects here described, comprising:
a main body destined to contain and/or support at least temporarily at least a device (1) for collecting and transferring samples of biological material;
a graduated scale (4), comprising a plurality of measurement markings (4i) configured to, and destined to, supply an at least visual and/or tactile indication of a measurement of a distance; said container (9) being configured to allow a measurement of a distance (D) between a first portion and a second portion of the body of a subject (20), preferably a distance (D) between an ear (10), in particular the tragus or lobe or opening of the ear canal (10t), and an opening or end of a naris (11) of a subject (20) and/or between an ear (10), in particular the tragus or lobe or opening of the ear canal (10t), and the philtrum (12) of the subject (20) by said graduated scale (4).

According to the present disclosure is also described a container (9) for a device (1) for collecting and transferring samples of biological material, preferably according to one or more of the aspects here described, comprising:
a main body destined to contain and/or support at least temporarily at least a predefined portion of the device (1) for collecting and transferring samples of biological material, in particular the collection element (3);
a graduated scale (4), comprising a plurality of measurement markings (4i) configured to, and destined to, supply an at least visual and/or tactile indication of a measurement of a distance; said container (9) being configured to allow a measurement of a distance (D) between a first portion and a second portion of the body of a subject (20), preferably a distance (D) between an ear (10), in particular the tragus or lobe or opening of the ear canal (10t), and an opening or end of a naris (11) of a subject (20) and/or between an ear (10), in particular the tragus or lobe or opening of the ear canal (10t), and the philtrum (12) of the subject (20) by said graduated scale (4).

According to another non-limiting aspect, the container (9) has at least a closed operative configuration wherein it is configured to contain said at least a device (1) in a condition of substantial sterility and/or of air insulation with respect to an outer environment, said container (9) being disposable and/or destined to contain a single device (1) and/or being pocket-shaped and/or being substantially flexible and/or foldable, and/or comprising a first element, preferably a cover, and a second element, preferably a base portion, removably connectible to said cover.

According to another non-limiting aspect, the container (9) has an open operative configuration wherein it allows to access the device (1).

According to another non-limiting aspect, at least part of said container (9) is realized in a natural material and/or in a plastic material.

According to another non-limiting aspect, at least part of said container (9) is substantially rigid, optionally self-supporting.

According to another non-limiting aspect, the container (9) comprises a substantially transparent or translucid portion, and wherein the graduated scale (4) is at least partially positioned in correspondence of said substantially transparent or translucid portion.

According to another non-limiting aspect, said substantially transparent or translucid portion is part of the main body and/or of the auxiliary body.

According to another non-limiting aspect, said substantially transparent or translucid portion is realized in plastic material.

According to another non-limiting aspect, the graduated scale (4) is positioned at an end position, in particular in substantial correspondence of a side of said container (9).

According to another non-limiting aspect, the container (9) comprises a predefined opening portion, visually different and/or weakened and/or pre-cut, said predefined opening portion being destined to allow a direct access to said gripping portion (2i).

According to another non-limiting aspect, on said container (9), the graduated scale (4) is at least partially etched, and/or realized through recesses or concavities, on said main body and/or is at least partially in relief on said main body and/or is fixed, impressed, molded, co-molded or painted on said main body, and/or wherein said graduated scale (4) is realized in a material at least partially different with respect to the material with which is realized said main body, in particular wherein the main body presents at least a first color and wherein at least part of said graduated scale (4) presents at least a second color different with respect to said first color.

According to another non-limiting aspect, the container (9) comprises an auxiliary body, operatively connected to the main body and optionally removable with respect to the main body.

According to another non-limiting aspect, the graduated scale (4) is positioned in substantial correspondence of at least one between the main body and/or the auxiliary body.

According to another non-limiting aspect, the auxiliary body is configured and specifically destined to be removed, optionally ripped, from the main body.

According to another non-limiting aspect, the container (9) comprises an identification code, in particular a visual and/or alphanumeric code, configured to identify, optionally univocally, said container (9) and/or said device (1).

According to another non-limiting aspect, the identification code is tag-shaped.

According to another non-limiting aspect, the identification code is arranged in correspondence of at least one between said main body and said auxiliary body.

According to another non-limiting aspect, the graduated scale (4) extends in substantially axial direction.

According to the present disclosure it is also described a kit comprising:
a device (1), in particular a device for collecting and transferring samples of biological material, optionally according to one or more of the aspects here described;
a container (9) according to one or more of the aspects here described.

According to another non-limiting aspect, the graduated scale (4) of said container (9) and the graduated scale (4) of the device (1) are quantitatively, in particular metrically, corresponding.

According to another non-limiting aspect, the kit comprises a reagent configured to determine the presence of viruses and/or of a bacterium, in particular of coronavirus.

According to another non-limiting aspect, said coronavirus comprises the SARS-CoV-2.

According to another non-limiting aspect the kit comprises a test card for viruses and/or bacterium presence, in particular of coronavirus.

According to the present disclosure it is described a method of determination of a correct depth of insertion of a device (1) for collecting and transferring samples of biological material, preferably according to one or more of the aspects here described, comprising:
a step of determination or measurement of a distance (D) between a first portion and a second portion of the body of a subject (20), preferably a step of determination of a distance (D) between an ear (10), in particular the tragus or lobe or opening of the ear canal (10t), and an opening or an end of a naris (11) of a subject (20) and/or between an ear (10), in particular the tragus or lobe or opening of the ear canal (10t), and the philtrum (12) of the subject (20), said step of determination or measurement being carried out through a measuring instrument, preferably by a container (9) according to one or more of the aspects here described and/or by a device (1) for collecting and transferring samples of biological material, optionally according to one or more of the aspects here described,
a detection or reporting of said distance (D), or of a measurement correlated in predetermined way to said distance (D), on a device (1) for collecting and transferring samples of biological material, and preferably on a device (1) for collecting and transferring samples of biological material according to one or more of the aspects here described, and in particular on a graduated scale (4) of said device (1), in order to determine an end stroke position for the insertion of said device (1) inside a cavity of the body of the subject (20),
a step of grasping of the device (1) by a user destined to allow, and/or preliminary to, an introduction of the collection element (3), and optionally of at least part of the elongated body (2), inside said cavity of the body of the subject (20), in particular inside a nasal and/or nasopharyngeal cavity of the subject (20), according to an insertion depth corresponding or correlated to said distance (D) and/or not further than said end stroke position.

According to another non-limiting aspect, the step of detection of said distance (D) comprises a step of positioning of the graduated scale (4) of said device (1) or of the graduated scale (4) of the container (9) in substantial proximity of a portion of the body of the subject (20), so that a first measurement marking (4i) of the graduated scale is aligned to said first portion or to said second portion of the body of the subject (20), and comprises a step of detection of said distance (D) by comparison with another measurement marking (4i) of the graduated scale, for quantitatively determining the measurement of said distance.

According to another non-limiting aspect, the method comprises step of reporting of the measurement of said distance (D) from graduated scale (4) of the container (9) to the graduated scale (4) of the device (1).

According to another non-limiting aspect, the method comprises a step of reporting of the measurement of said distance (D) from graduated scale (4) of the container (9) to the device (1), optionally non-graduated, by attachment by the user of a reference mark, such as for example a line realized by a felt-tip pen or similar.

According to another non-limiting aspect, the method abode described explicitly excludes the introduction of the device (1) within said cavity of the body of the subject (20), in particular by excluding the introduction of the device (1) inside the nasal cavity and/or the nasopharynx.

According to another non-limiting aspect, the method comprises a step of introduction of the device (1) inside a cavity of the body of the subject (20), preferably inside the nasal cavity and/or the nasopharynx.

According to another non-limiting aspect, said step of grasping comprises the grasping of the elongated body (2) in substantial correspondence of a predetermined measurement marking (4i) corresponding to said measured distance (D).

According to another non-limiting aspect, the method comprises the introduction of the device (1) within said portion of the body of the subject (20), the method comprising the carrying out of the step of grasping for at least part of, preferably all the, introduction of the device (1) within said cavity of the body of the subject.

According to another non-limiting aspect, the introduction is a substantially axial introduction and, optionally, comprises a substantially axial translation along a direction detected by said elongated body (2) and/or a rotation, preferably the rotation taking place along an axis substantially coinciding with an axis detected by said elongated body (2).

According to the present disclosure it is also described the use of the device (1) according to one or more of the aspects here described for the collection of samples of biological material from a nasal cavity.

In particular, according to the present disclosure it is also described the use of the device (1) according to one or more of the aspects here described for the carrying out of presence tests of bacteria and/or viruses, in particular for the carrying out of presence tests of coronavirus, optionally of SARS-CoV-2 type.

### FIGURES

The object of the present disclosure will be described in the detailed description, which refers to some preferred embodiments of device 1. In particular, these preferred embodiments are described with reference to the attached figures; a brief description of the figures is provided hereinafter.

FIG. 1 shows a lateral view of a device according to the present disclosure.

FIG. 2 shows a profile view of a human face from which it is possible to determine a distance D between the nose and the ear, which is associated to an introduction depth of the device of FIG. 1.

FIG. 3 shows a sectional view of part of the device object of the present disclosure, in a specific embodiment.

FIG. 4 shows a non-limiting embodiment of a container for a device according to the present disclosure.

FIG. 5 shows a specific embodiment of the device object of the present disclosure, wherein a plurality of measurement markings of a graduated scale are etched or recessed.

FIG. 6 shows a specific embodiment of the device object of the present disclosure, wherein the plurality of measurement markings protrudes outside the elongated body.

FIG. 7 shows a specific embodiment of the device object of the present disclosure, wherein the plurality of measurement markings is of a color different with respect to the color of the elongated body.

FIG. 8 shows a specific embodiment of the device object of the present disclosure, wherein the plurality of measurement markings is arranged, partially, obliquely with respect to a main development direction of the elongated body.

### DETAILED DESCRIPTION

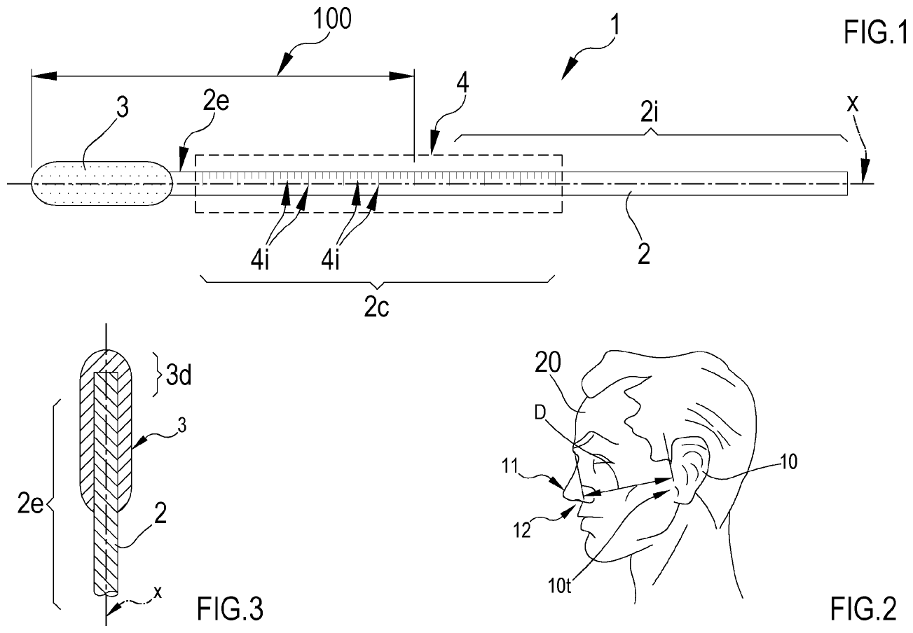

With reference to FIG. 1, with reference number 1 is indicated as a whole a device 1 for collecting and transferring samples of biological material. For the purposes of the present disclosure, for “biological material” it is intended a material, for example wet material, in particular and not limiting thereto fluid and/or liquid, containing at least one between a secretion of a tissue, a tissue sample, tissue cells, blood, etc. . . . , of a human or animal being.

In a preferred embodiment, the device 1 is mainly destined to allow the collection of biological samples from a nasal cavity, in particular from a portion of mucosa of the nasal cavity and/or nasopharynx. The device 1 here described can be advantageously used, for example, for the carrying out of screening and/or test of presence of coronavirus, in particular, and non-limiting thereto, of virus of SARS-CoV2 type (base variant and its subsequent variants). The device 1 here described therefore realizes a so-called nasopharyngeal swab.

The device 1 can be part of a kit which comprises, further than the device 1 itself, also a container 9, that will be better described hereinafter. The container 9 forms in turn an object of the present disclosure. Said kit can conveniently comprise a card for carrying out a test of presence of at least one between a virus and/or a bacterium, in particular a coronavirus, and more in particular for carrying out a test of presence of the SARS-CoV-2 antigen, and a reagent. Conveniently, said card and said reagent can be contained inside said container 9. Preferably, said kit comprises also an instruction leaflet necessary to allow a user to understand—where there are doubts—how to use the kit itself, in particular how to make a correct measurement of a distance D between a first and a second portion of the human body, in order to introduce the device 1 into a human body cavity, in particular the nasal cavity, for a correct introduction depth. Such an instruction leaflet can be conveniently replaced by a suitable instruction printout executed directly on the part of the container 9.

The device 1 comprises an elongated body 2, mainly and at least partially extending along a main substantially axial development direction (axis) X.

The elongated body 2 is preferably realized in plastic material; this technical feature allows to have an elongated body 2 that is extremely economical to produce. The elongated body 2 is substantially flexible, in order to adapt at least partially to a curvature of the nasal cavity, the naris and/or the nasopharynx. For example, the elongated body 2 can have a substantially circular transversal section, and develop along the axis X with a substantially constant diameter, e.g. and not limiting thereto comprised between 1.5 mm and 3 mm, more preferably comprised between 2 mm and 2.5 mm. When not of circular transversal section, the shape of the transversal section can be enclosed in a circumference whose diameter respects the limits described above.

This should not be understood in a limiting way, since in an alternative embodiment, the elongated body 2 can have a diameter, or more generally a transversal section, of non-constant area as one moves along the axis X. When realized in plastic material, the elongated body 2 can be realized by moulding, in particular by injection moulding or co-moulding. In an embodiment, the elongated body 2 can be realised by a 3D printing process.

The 3D printing process can be conveniently carried out by using a polymeric plastic material, for example and not limiting thereto, such as polyester type, for example PBT, or PEEK, or PPS, or polycarbonate, or ABS, or PVC, or PMMA. It is to be understood that where in the present disclosure reference is generically made to a plastic material, the specific types of plastic material described herein in connection with 3D printing of the elongated body can be equivalently used, alternatively or in addition to other specifically described types of material.

The elongated body 2 comprises at least a gripping portion 2i destined to be grasped by a user and an end portion 2e; the end portion 2e is opposed with respect to the gripping portion 2i.

The user can be a health professional, in particular a doctor or a paramedic, for example a nurse, or a pharmacist. The specific profession of the person referred to herein as “user” is not to be understood in a limiting way; the differentiation between “user” and “subject” is provided merely for the purpose of descriptive clarity. The user can also be the subject 20, in particular when the latter performs a self-test via said device 1.

Superficially, the elongated body 2, in particular in correspondence of the gripping portion 2i, can be equipped with elements (for example knurling) destined to increase the grip with the fingers and/or palm of the user's hand. In an embodiment, at least part of the gripping portion 2i can be provided with a rubberized overlay.

The device 1 comprises also a collection element 3 connected to the elongated body 2 in substantial correspondence of the end portion 2e. The collection element 3 is configured and specifically destined to allow to absorb and/or retain a quantity of a sample of biological material, in particular of a secretion which develops in the in the upper airways of a subject. The collection element 3 is configured to be introduced inside the nasal cavity and/or nasopharynx of a subject.

The elongated body 2 is fixed with respect to the collection element 3; in particular this means that the elongated body 2 does not slide with respect to the collection element 3.

In a non-limiting embodiment, the collection element 3 is realized above a portion, in particular above the end portion 2e, of the elongated body 2.

For the purposes of the present disclosure, for “nasal cavity” it is intended the portion of human body that starts from the nose tip and connects to the nasopharynx.

For the purposes of the present disclosure, for “nasopharynx” it is intended the upper portion of the pharynx that extends from the base of the skull until the upper surface of the soft palate. The nasopharynx includes the space between the inner narises and the soft palate, and lies above the oral cavity. The nasopharynx comprises, in particular, the adenoid portion. The nasopharynx is surrounded by the Waldeyer ring.

Preferably, but non limiting thereto, the collection element 3 is configured and specifically destined to allow the collection of at least 40 ml, or 50 ml, or 70 ml, or 100 ml of fluid. This ensures a collection of fluid sufficient for carrying out in a reliable way further tests, in particular diagnostic type tests and more in particular nucleic acid amplification tests (NAAT) with a reverse polymerase chain reaction process. This makes the collection element 3 suitable for the collection of samples for carrying out screening and/or test of presence of coronavirus.

In a specific embodiment, the collection element 3 can be pre-inhibited or impregnated with a substance, in particular with a reagent.

In a preferred embodiment, the collection element 3 comprises a plurality of fibers provided with hydrophilic capability. The collection element 3 could be for example a flocked swab, a wound fiber swab, a sponge swab or a swab in another absorbing material. In case of a flocked swab, the flocking process is such as to allow the deposit of fibers in an electrostatic field, that allows to deposit the fibers in an ordered way, perpendicularly to the surface of the end portion 2e of the elongated body.

In an embodiment, fibers are selected from a list of materials comprising synthetic and natural materials, and in particular from a list of materials comprising at least one between: rayon, polyester, polyamide, carbon fiber, alginate, cotton, silk or mixtures of them, sponge.

In a preferred embodiment, but non limiting thereto, at least part of the end portion 2e protrudes inside the collection element 3; more in particular, the Applicant has conceived a specific embodiment wherein the end portion 2e extends until in substantial correspondence of the distal end 3d of the collection element 3. The latter surrounds then at least part of said end portion 2e. This particular embodiment is represented in FIG. 3.

In order to promote a uniformity of collection of the biological sample, in an embodiment the collection element 3 is preferably but not limiting thereto of cylindrical and/or truncated cone shape; in a non-limiting embodiment, the distal end 3d is substantially rounded and this contributes to reduce the risk of inadvertently injuring or damaging part of the nasal cavity and/or nasopharynx during the introduction and/or extraction of the device 1.

In an embodiment, then, the collection element 2 completely surrounds the elongated body 2 in correspondence of the end portion 2e.

The elongated body 2 comprises a graduated scale 4 comprising a plurality of measurement markings 4i configured to supply an at least visual indication of a distance with respect to at least one between the end portion 2e and the collection element 3. In an embodiment the graduated scale 4 comprises a first, a second and a third measurement marking 4i, and two by two these measurement markings are equally spaced. The graduated scale 4 extends preferably along a substantially axial direction.

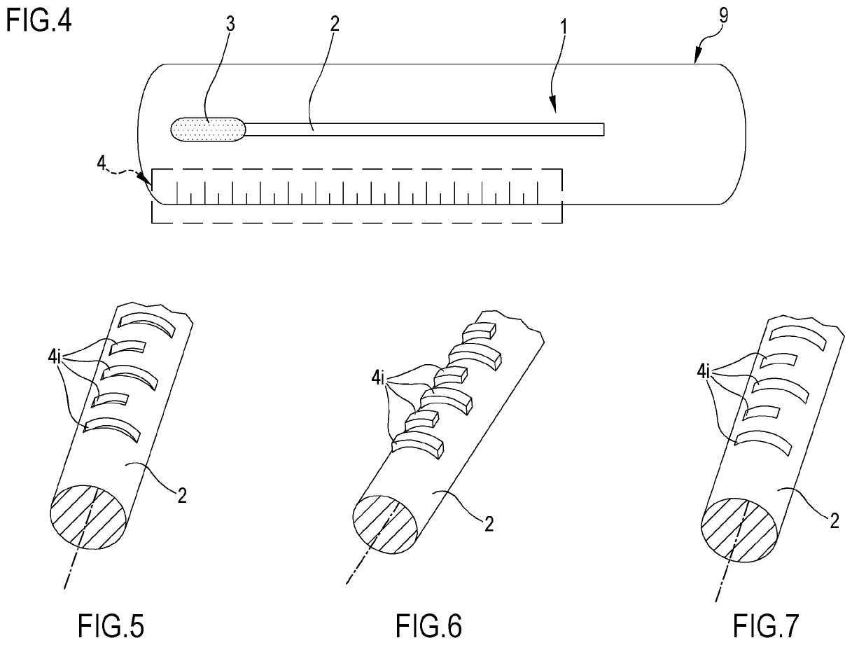

As shown in FIG. 1, and more in detail in the various embodiments shown in FIGS. 4-7, the graduated scale 4 is configured and destined to supply an at least visual indication of a preferred introduction depth (indicated with reference 100) at least of the collection element 3 inside the nasal cavity and/or nasopharynx, in particular in relation to a conformation of the nasal cavity and/or nasopharynx and/or in relation to a distance D (shown in FIG. 2) between an ear 10, in particular the tragus or lobe or opening of the ear canal (10t), and an opening of the naris 11 of a subject 20 and/or between an ear 10, in particular the tragus or lobe or opening of the ear canal 10t, and the philtrum 12 of the subject 20.

The graduated scale 4 allows the quantitative identification of the distance between at least part of the plurality of measurement markings 4i and said at least one between said end portion 2e and said collection element 3. In particular, the graduated scale 4 can be one according to the decimal metric system, and express measurements in cm or in mm, or according to the anglosaxon system (United States Customary System, USC), by expressing for example measurements in inches. In an embodiment, the graduated scale 4 allows the quantitative identification of the distance of the plurality of measurement markings 4i and the tip of the collection element 3.

The fact that the elongated body 2 is fixed with respect to the collection element 3, determines that also the graduated scale 4, and in particular the plurality of measurement markings 4i, is in a fixed position with respect to the collection element 3.

In an embodiment, the device 1 is free from movable parts; this makes it particularly easy to realize and use, and reduces the risk of improper use.

Although this should not be understood in a limiting way, in an embodiment, the graduated scale 4 possesses at least a first measurement marking 4i positioned in substantial proximity of the collection element 3, in particular in substantial contact with the latter. This makes it possible to introduce the device 1 for very short depths 100.

The Applicant has in particular observed that the distance D between an ear 10, in particular the tragus or lobe or opening of the ear canal 10t, and an opening, or end, of the naris 11 of a subject 20 and/or between an ear 10, in particular the tragus or lobe or opening of the ear canal 10t, and the philtrum 12 of the subject 20 varies with the variation of the age of the subject and is substantially comprised between 4 cm, more preferably 4.5 cm and 10.5 cm, more preferably 10 cm. In particular it has been observed that between 4 and 5 years old, said distance D is substantially equal to 5 or 6 cm, and increases substantially proportionally until 11-12 years; in correspondence of this age the distance D stabilizes around a mean value of 8-10 cm. Consequently, the graduated scale 4 extends such as to determine lengths until at least 10 cm.

In order to allow a correct grasping of the elongated body 2, and in order also to allow to reach a correct introduction of the device 1 until reaching the collection element 3 the rear mucosa of the nasopharynx, preferably the device 1 presents a length at least equal to 12 cm, and preferably at least equal to 13 cm or 14 cm. Thanks to this aspect, in particular together with the presence of the graduated scale 4, is possible to produce the elongated body 2 in a single size, by limiting then the production costs, and using said elongated body 2 for reaching, without damaging and/or hurting and/or causing discomfort, substantially the rear mucosa of the nasopharynx both in adults and in children.

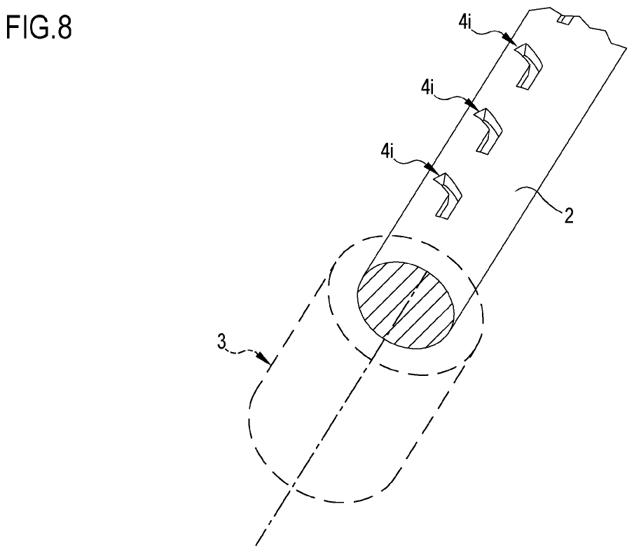

The plurality of measurement markings 4i comprises at least a first and a second measurement marking 4i reciprocally spaced for a predefined distance along the main development direction X. At least a first and a second measurement marking 4i extend at least partially, preferably mainly, along a substantially oblique direction, optionally along a substantially orthogonal direction, with respect to the main development direction X. In particular FIGS. 4-7 show embodiments wherein measurement markings 4i extend substantially orthogonally with respect to the main development direction, whereas the FIG. 8 shows an embodiment wherein the measurement markings 4i extend substantially obliquely and partially parallel to said main development direction X.

In FIG. 4 it is shown an embodiment wherein the graduated scale 4 is at least partially etched on the elongated body 2, and realizes therefore at least a recess, in particular a plurality of recesses, in correspondence of measurement markings 4i. The etching process is not described here because it is known; however, in an embodiment the etching is actually a concavity deriving from a molding or 3D printing process of the elongated body 2.

In FIG. 5 it is shown an embodiment wherein the graduated scale 4 is in relief on said elongated body 2. Said graduated scale 4 can be fixed and/or impressed in any known method. Also in this case, the etching can be a convexity deriving from a molding process (in particular co-molding) or 3D printing of the elongated body 2.

The Applicant in particular observes that the graduated scale 4 can therefore be realized integrally with the elongated body 2, then as a single piece. It is observed also that the elongated body 2, thanks to the molding process or 3D printing can be integral, then realized as a single piece. This contributes to increase the robustness and uniformity of mechanical properties along its extension.

In FIG. 6, and in FIG. 7, it is shown an embodiment wherein the graduated scale 4 is painted on said elongated body 2. In particular, the elongated body 2 possesses at least a first color and at least part of the graduated scale 4 possesses a second color different with respect to the first color.

In figures from 5 to 8, the transversal section of the elongated body 2 is substantially circular and the measurement markings 4i extend along an arc of circumference smaller than the circumference itself. This should not be understood in a limiting way, since in an alternative embodiment the measurement markings 4i are developed along the entire circumference. When the elongated body 2 assumes a shape different with respect to the circular one, it will therefore be clear that the measurement markings 4i can develop along at least part of the perimeter or transverse development of the elongated body 2, or alternatively they can develop along the entire perimeter or transverse development of the elongated body 2.

It is observed, in particular, that the graduated scale 4 can be realized in a material at least partially different with respect to the material with which the elongated body 2 is realized.

FIG. 3 shows a container 9 for the device 1, or—more generally—for any device for collecting and transferring samples of biological material. The container 9 detects a substantially rigid main body which is configured and specifically destined to contain at least part of the device 1 here described. In FIG. 3, the container 9 is shown with a substantially rectangular shape which must not be intended in a limiting way. The container 9 can be realized as a single piece or two pieces; in this last case it comprises a first and a second portion, in particular and in a non-limiting way a base and a cover removable with respect to the base.

The container 9 can be configured to house the whole device 1, in particular to enclose the whole device 1. In an alternative embodiment, the container 9 can be configured and specifically destined to allow to contain or house only part of the device 1, preferably only the collection element 3. The latter, in fact, represents the most sensitive part of the device 1, in particular the part whose contamination should be attentively avoided.

The container 9 can comprise an auxiliary body, operatively united to the main body. In an embodiment, the secondary body can be disconnected from the main body. In particular said secondary body can be provided with a hole, or assume a ribbon or cord shape, in order to allow to attach or fix the main body in a predetermined position. The auxiliary body can be configured to be removed, and in particular ripped, from the main body. The secondary body can be also flexible, and contain an identification tag containing for example a visual (i.e. image-shaped) or alphanumeric code, identifying (for example univocally identifying) the specific container 9 and/or the device 1 here contained.

The container 9 can be realized in any material between a synthetic or natural material, for example cardboard or plastic. In this latter case, the container 9 can be molded or 3D printed. In an embodiment the container 9 can be realized substantially pocket-shaped, and be substantially flexible and/or foldable. A particular embodiment of the flexible and/or foldable container provides for the possibility to carry out an edge, so that a user can access the elongated body 2 by keeping at least temporarily, in use, the collection element 3 at least partially covered and/or covered by at least part of the container itself. Alternatively, or in combination with the above, at least part of the container 9 can be substantially rigid, and in particular can be self-supporting.

In a non-limiting embodiment, the container 9 can comprise a weakened and/or pre-cut and/or visually different portion, in correspondence thereof said container can be opened. This weakened and/or pre-cut and/or visually different portion, can be a portion substantially comprised between the main body and the auxiliary body and/or can be effectively part of the main body and/or of the auxiliary body.

Conveniently, this portion can be arranged in a predefined positional relationship with respect to the device 1, so that it is determined the possibility, at the opening of the container 9, to directly access the gripping portion 2i. Thanks to this aspect, it is reduced the risk of involuntary contamination of the element 3.

On the main body of the container 9 there is a graduated scale 4 comprising too a plurality of measurement markings 4i configured to allow the quantitative identification of the distance D between an ear 10, in particular the tragus or lobe or opening of the ear canal 10t, and an opening, or end, of the naris 11 of a subject 20 and/or between an ear 10, in particular the tragus or lobe or opening of the ear canal 10t, and the philtrum 12 of the subject 20. Also in this case the graduated scale 4 extends preferably along a substantially axial direction.

In an alternative embodiment, the graduated scale 4 is positioned on the auxiliary body. In another alternative embodiment, part of the graduated scale 4 lies on the main body and part of the graduated scale 4 lies on the auxiliary body.

Preferably, but non limiting thereto, the graduated scale 4 is positioned in substantial correspondence of a side of the container 9, in order to facilitate the measurement of said distance D.

The Applicant in particular observes that the measurement of said distance D through the container 9 allows to reduce the risk of contaminations of the collection element 3 with surfaces, for example skin and/or hair, external with respect to the nasal cavity.

Similarly to what it has been described for the graduated scale of the device 1, also in case of the container 9 the graduated scale 4 is at least partially etched, and/or realized through recesses or concavities, on said main body and/or is at least partially in relief on said main body and/or is fixed, impressed, molded (in particular, 3D printed), co-molded or painted on the main body, and/or the graduated scale 4 is realized in a material at least partially different with respect to the material with which the main body is realized.

In another embodiment, the graduated scale 4 is painted on said container 9. For this reason, in a portion where it is present the graduated scale 4 the container 9 possesses at least a first color and at least part of the graduated scale 4 possesses a second color different with respect to the first color.

In a specific and non-limiting embodiment, at least in correspondence of the main body (or, optionally of the auxiliary body), and in particular in substantial correspondence of the graduated scale 4, the container 9 can advantageously be of transparent or translucid type. The portion of plastic or translucid container is advantageously realized in plastic material. Thanks to this technical feature, a correct positioning of the graduated scale 4 between the nose and the ear of the subject 20 is facilitated.

In a non-limiting embodiment, the container 9 is configured and specifically destined to allow the maintenance of the device 1, in particular at least of the collection element 3, substantially sterile. Although the container 9 can be destined to contain a plurality of devices 1, in a specific embodiment the container 9 is a container for a single device 1, retained in a sealed, disposable pocket, that allows to reduce the cross-contamination risk. In an embodiment, therefore, the container 9 comprises at least a closed operative configuration wherein the at least one device 1 is kept in a sterile condition, and is hermetically insulated from the outer environment. To this closed operative configuration is opposed an open operative configuration, characterized for example by the opening of the cover (if present) or by the ripping of at least a portion of said pocket.

The present disclosure refers also to a method of determination of a correct depth of insertion of a device 1 for collecting and transferring samples of biological material, preferably according to what has been here described. Said method allows to determine a correct depth of insertion of the device 1.

A first step is a step of determination or measurement of the distance D between a first portion and a second portion of the body of a subject 20; this step is preferably a step of determination of a distance D between an ear 10, in particular the tragus or lobe or opening of the ear canal 10t, and an opening or an end of a naris 11 of a subject 20 and/or between an ear 10, in particular the tragus or lobe or opening of the ear canal 10t, and the philtrum 12 of the subject 20.

It is observed in particular that the step of determination or measurement is performed through a measuring instrument, preferably through a container 9, in particular according to what has been here described and/or through a device 1, in particular according to what has been here described.

A subsequent step is a step of detection or reporting of the distance D on a device 1 for collecting and transferring samples of biological material, and preferably on a device according to what is described here, and in particular on the graduated scale 4 of said device 1.

The detection is carried out in order to determine an end stroke position for the insertion of said device 1 inside a cavity of the body of the subject 20.

A step of grasping of the device 1 follows then by a user (preferably, but non limiting thereto, different from the subject) destined to allow, and/or preliminary to, an introduction of the collection element 3, and optionally of at least part of the elongated body 2, inside the cavity of the body of the subject 20, in particular inside the nasal cavity and/or nasopharynx of the subject 20, according to an insertion depth corresponding or correlated to said distance D and/or not further than said end stroke position.

In the above described method it is observed in particular that if the container 9 has been used, the value of distance D read on the graduated scale 4 of the container shall be reported on the corresponding value of the graduated scale 4 of the device 1.

The step of grasping comprises the grasping of the elongated body 2 in substantial correspondence of a predetermined measurement marking 4i corresponding to said distance, and this grasping is particularly destined to allow an introduction of the at least a collection element 3, and optionally of at least part of the elongated body 2 inside the nose for a portion lower or equal to the portion determined by said predetermined measurement marking 4i. Substantially, therefore, the fingers of the user become an end stroke stop that determines the correct (and maximum) introduction depth. If the subject 20 on which a collection is carried out manifests discomfort or pain, the user can clearly limit the introduction of the device 1 to a depth lower with respect to the one allowed by the determination previously performed.

The Applicant observes that although the present detailed description makes specific reference to a distance D detected in substantial correspondence of the head of the subject, this should not be intended as a specific and limiting purpose of use of the device 1 object of the present disclosure. In particular, in fact, said device 1 can be favourably destined to allow the collection of samples of biological material from another cavity of the human or animal body, in particular from the ear, mouth, rectum or urethra. Accordingly, the dimensions, in particular a length and/or a transversal section of the collection element 3, or a length of the elongated body 2 or even a transversal section thereof, can be conveniently adapted according to the specific application. The extension of the graduated scale 4 can also be varied accordingly.

It is observed finally that even if the subject 20 has mainly been intended as a “human subject”, this should not be understood in a limiting way; in fact, the subject 20 may also be an animal, such as a pet. It is therefore clear that what is the object of the present disclosure must not necessarily be considered to be exclusively directed to applications on human beings, since said device 1, container 9 and method can equally be intended for veterinary applications.

The invention is not limited to the embodiments shown in the attached figures; for this reason, the reference marks appearing in the following claims are provided for the sole purpose of increasing the intelligibility of the claims, and should not be intended in a limiting way.

Finally, it is clear that additions, modifications or variants that are obvious to an expert in the art can be applied to the object of the present disclosure, without for this departing from the scope of protection provided by the claims.

1. Container for a device for collecting and transferring samples of biological material, comprising:
a main body destined to contain and/or support at least temporarily at least a device for collecting and transferring samples of biological material;
a graduated scale, comprising a plurality of measurement markings realized according to a predetermined distance measurement unit and configured to, and destined to, supply an at least visual and/or tactile indication of a measurement of a distance; said container being configured to allow a measurement of a distance between a first portion and a second portion of the body of a subject, preferably a distance between an ear, in particular the tragus or lobe or opening of the ear canal, and an opening or end of a naris of a subject and/or between an ear, in particular the tragus or lobe or opening of the ear canal, and the philtrum of the subject by said graduated scale.

2. Container according to claim 1, having at least a closed operative configuration wherein it is configured to contain said at least a device in a condition of substantial sterility and/or of air insulation with respect to an outer environment, said container being disposable and/or destined to contain a single device and/or being pocket-shaped and/or being substantially flexible and/or foldable, and/or comprising a first element, preferably a cover, and a second element, preferably a base portion, removably connectible to said cover.

3. Container according to claim 1, comprising a substantially transparent or translucid portion, and wherein the graduated scale is at least partially positioned in correspondence of said substantially transparent or translucid portion.

4. Container according to claim 1, wherein said graduated scale is positioned at an end position, in particular in substantial correspondence of a side of said container, and/or comprising a predefined opening portion, visually different and/or weakened and/or pre-cut, said predefined opening portion being destined to allow a direct access to said gripping portion.

5. Container according to claim 1, wherein the graduated scale is realized on said main body and/or is at least partially etched, and/or realized through recesses or concavities, on said main body and/or is at least partially in relief on said main body and/or is fixed, impressed, molded, co-molded or painted on said main body, and/or wherein said graduated scale is realized in a material at least partially different with respect to the material with which is realized said main body, in particular wherein the main body presents at least a first color and wherein at least part of said graduated scale presents at least a second color different with respect to said first color.

6. Container according to claim 1, comprising an auxiliary body, operatively connected to the main body and optionally removable with respect to said main body;
wherein the graduated scale is positioned in substantial correspondence of at least one between the main body and/or the auxiliary body.

7. Container according to claim 1, comprising an auxiliary body, operatively connected to the main body and optionally removable with respect to the main body,
and wherein the graduated scale is positioned in substantial correspondence of at least one between the main body and/or the auxiliary body and/or wherein the auxiliary body is configured and specifically destined to be removed, optionally ripped, from the main body.

8. Container according to claim 1, comprising an identification code, in particular a visual and/or alphanumeric code, configured to identify, optionally univocally, said container and/or said device, optionally wherein the identification code is tag-shaped.

9. Device for collecting and transferring samples of biological material, comprising:
an elongated body, at least partially extending along a main substantially axial development direction and comprising at least a gripping portion destined to be grasped by a user and at least an end portion;
a collection element connected to the elongated body in substantial correspondence of said end portion and configured to collect and/or absorb a quantity of a sample of biological material present in a cavity of the body of a subject, at least the collection element being configured to be introduced inside a cavity of the body of the subject, for example to be introduced inside the nasal cavity and/or the nasopharynx of the subject,
wherein the elongated body comprises a graduated scale provided with a plurality of measurement markings realized according to a predetermined distance measurement unit, said measurement markings being configured to, and destined to, provide an at least visual and/or tactile indication of a measurement of a distance with respect to said end portion of the elongated body and/or to a predefined portion of said collection element, preferably at the end of said collection element opposed to the gripping portion.

10. Kit comprising:
a device for collecting and transferring samples of biological material preferably according to claim 9;
a container according to claim 1.
optionally wherein the graduated scale of said container and the graduated scale of the device are quantitatively, in particular metrically, corresponding and/or are realized according to a same predetermined distance measurement unit.

11. Method of determination of a correct depth of insertion of a device for collecting and transferring samples of biological material, comprising:
a step of determination or measurement of a distance between a first portion and a second portion of the body of a subject, preferably a step of determination of a distance between an ear, in particular the tragus or lobe or opening of the ear canal, and an opening or an end of a naris of a subject and/or between an ear, in particular the tragus or lobe or opening of the ear canal, and the philtrum of the subject, said step of determination or measurement being carried out through a measuring instrument, preferably a container according to one or more of claim 1 and/or by a device for collecting and transferring samples of biological material, optionally the device according to claim 9,
a detection or reporting of said distance, or of a measurement correlated in predetermined way to said distance, on a device for collecting and transferring samples of biological material, in order to determine an end stroke position for the insertion of said device inside a cavity of the body of the subject, said step of detection or reporting being preferably carried out on a device for collecting and transferring samples of biological material according to claim 9, and in particular on the graduated scale of said device according to claim 9,
a step of grasping of the device by a user destined to allow, and/or preliminary to, an introduction of the collection element, and optionally of at least part of the elongated body, inside said cavity of the body of the subject, according to an insertion depth corresponding or correlated to said distance and/or not further than said end stroke position, optionally wherein said cavity is a nasal and/or nasopharyngeal cavity of the subject.
